(19) United States
(12) Patent Application Publication
(10) Pub. No.: US 2025/0288261 A1
(43) Pub. Date: Sep. 18, 2025

(54) ALIGNMENT OF CT IMAGE DATA FOR CARDIAC EXAMINATION

(71) Applicant: Siemens Healthineers AG, Forchheim (DE)

(72) Inventors: Thomas ALLMENDINGER, Forchheim (DE); Bernhard SCHMIDT, Fuerth (DE)

(73) Assignee: Siemens Healthineers AG, Forchheim (DE)

(21) Appl. No.: 19/080,093

(22) Filed: Mar. 14, 2025

(30) Foreign Application Priority Data
Mar. 15, 2024 (DE) 10 2024 202 451.1

(51) Int. Cl. A61B 6/03; A61B 6/00; G06T 5/20; G06T 11/00

(52) U.S. Cl. CPC A61B 6/032; A61B 6/481; G06T 5/20; G06T 11/006; G06T 2207/10081; G06T 2207/30048

(57) ABSTRACT

In a method for spectrally differentiated cardiac CT imaging, first spectrally differentiated CT projection measurement data relating to a heart is received in a first time interval, when a contrast agent is located in chambers and/or vessels of the heart. The first time interval comprises a diastole of the heart. Second spectrally differentiated CT projection measurement data relating to the heart is received in a second time interval, when the contrast agent is located in a muscle tissue of the heart. The second time interval includes a systole of the heart. First monoenergetic image data is calculated for a first energy value based on the first spectrally differentiated CT projection measurement data and second monoenergetic image data is calculated for the first energy value based on the second spectrally differentiated CT projection measurement data.

### CROSS-REFERENCE TO RELATED APPLICATION(S)

The present application claims priority under 35 U.S.C. § 119 to German Patent Application No. 10 2024 202 451.1, filed Mar. 15, 2024, the entire contents of which is incorporated herein by reference.

### FIELD

One or more embodiments of the present invention relate to a method for spectrally differentiated cardiac CT imaging. In addition, one or more embodiments of the present relate to an image generating device. Furthermore, one or more embodiments of the present relate to a computed tomography system.

### BACKGROUND

To clarify a potential risk caused by possible coronary heart disease, a CT scan (CT stands for computed tomography) of the heart is often used in routine clinical practice now. The most frequently used examinations are image recordings to determine a calcium score without contrast agent and coronary angiography, abbreviated to “CTA” (CTA stands for “CT Angiography”).

Both methods are primarily aimed at the visualization and examination of the coronary vessels and “ignore” the condition of the myocardium as another part of the heart which is also of diagnostic relevance. With the introduction of spectral CT systems, depending on the clinical issue, the acquisition is extended by a third scan, referred to as an ECV measurement, which is delayed by 3 to 5 minutes in relation to the coronary angiography and permits the fibrotic portion of the myocardium to be quantitatively estimated (ECV measurement: abbreviation for “Extra Cellular Volume” measurement).

In direct comparison to diagnosis based on magnetic resonance imaging, the exclusive use of computed tomography for the complete assessment of the heart only lacks a statement on the dynamics of the heart muscle or on an estimation of the change in the thickness of the heart muscle, i.e. a quantitative comparison between images taken in the systolic and in the diastolic cardiac phases. The systolic cardiac phase characterizes the tense state of the heart muscle in which the blood is pumped out of the ventricles into the pulmonary artery and the aorta, the heart muscle contracts and the heart wall is particularly thick. The diastolic cardiac phase characterizes the relaxed state of the heart muscle in which the ventricles are completely filled with blood and the heart is expanded to the maximum. The heart wall is particularly stretched and therefore particularly thin.

In principle, this function statement regarding the dynamic behavior of the heart is possible with a CT system, but until now has required the coronary angiography to be performed with a very wide ECG window (“ECG” stands for “electrocardiogram”), so that a reconstruction at the two characteristic times of systole and diastole of the cardiac cycle is possible. For example, such a combined CT scan requires a recording time of approximately 8 seconds, the use of a high dose being necessitated. A total of approximately 5 to 6 cardiac phases are required for one image during diastole and one image during systole, with diastole and systole alternating. The resulting radiation exposure is undesirably high. Furthermore, a third CT scan must take place after approximately three to five minutes in order to perform an ECV measurement.

Due to the significantly increased dose of radiation as a consequence, this procedure is usually avoided and a corresponding examination of the dynamics of the heart is carried out with the aid of magnetic resonance imaging.

### SUMMARY

At least one task is therefore to provide sufficient image data for an examination of the coronary vessels, an examination of the fibrotic portion of the myocardium and an examination of the dynamics of the heart muscle with the aid of CT imaging, wherein the radiation exposure should be lower than in a conventional procedure.

At least this object is achieved by a method for spectrally differentiated cardiac CT imaging of a patient as claimed, an image generating facility (also referred to as an imaging generating device) as claimed and a computed tomography system as claimed.

With the aid of spectrally differentiated projection measurement data, preferably spectrally resolved projection measurement data, for example, a purely diastolic acquisition can be offset against a systolic late acquisition so that an evaluation of the change in the wall of a heart of a patient is successfully possible and at the same time, this does not result in greatly increased radiation exposure of the patient.

In the method, according to an embodiment of the present invention, for spectrally differentiated cardiac CT imaging, first spectrally differentiated CT projection measurement data relating to a heart of a patient in a first time interval is received, a contrast agent mainly being located in the ventricles and the vessels of the heart during the first time interval, the first time interval comprising a diastole of the heart.

In particular, it may be provided that the first spectrally differentiated CT projection measurement data is acquired with the aid of a contrast agent. “Acquisition with the aid of a contrast agent” is to be understood as the recording of projection measurement data in the presence of a contrast agent in the respective examination area, i.e. in this case in the area of the heart of a patient. Spectrally differentiated CT projection measurement data is to be understood as projection measurement data which was either recorded with different spectra or recorded with spectral resolution. Photon-counting, spectrally resolving X-ray detectors are preferably used to generate such projection measurement data. Alternatively, different X-ray spectra can also be generated from one or more X-ray sources, either temporally or spatially differentiated accordingly. Typical systems for this are so-called dual-energy CT systems or split-filter CT systems.

In the method, according to an embodiment of the present invention, second spectrally differentiated CT projection measurement data which relates to the heart of the patient in a later second time interval is also received, the contrast agent mainly being located in the muscle tissue of the heart during the second time interval. The second time interval comprises a systole of the heart. In particular, it may be provided that the second spectrally differentiated CT projection measurement data is acquired with the aid of a contrast agent.

From a chronological point of view, the second time interval is preferably approximately 3 to 5 minutes after the first time interval. After this time, the contrast agent has moved from the large blood-bearing structures, i.e. the large vessels and the ventricles, into the muscle tissue, so that an ECV measurement can now be taken to examine the condition of the tissue. This measurement can be combined with the event of a systole so that this second measurement, in combination with the first measurement taken during a diastole, can also be used to determine the dynamic behavior of the heart muscle and to compare the thickness of the heart muscle in the diastolic phase and in the systolic phase.

Subsequently, first monoenergetic image data is calculated on the basis of the first spectrally differentiated CT projection measurement data for a first energy value. The calculation of monoenergetic image data, also referred to as “pseudo-monoenergetic image data”, is described in detail in Alvarez R. E. and Macovski A. “Energy-selective reconstructions in X-ray computed tomography”, Phys. Med. Biol. 21, 733-744 (1976).

When calculating the monoenergetic image data, it is crucial that the CT projection measurement data is spectrally differentiated or preferably spectrally resolved. Calculation includes a number of corrections, in particular of beam hardening effects, not only of water, but also of the contrast agent, in particular iodine. Furthermore, different spectral information can then be calculated either in the projection measurement data room, followed by reconstruction, preferably via filtered back projection, in order to obtain monoenergetic image data, or alternatively, different items of image data can first be reconstructed on the basis of different spectrally differentiated projection measurement data and then the reconstructed items of image data provided with different spectral information can be offset against each other in order to generate monoenergetic image data.

Likewise, second monoenergetic image data is calculated on the basis of the second spectrally differentiated CT projection measurement data for the first energy value. The monoenergetic image data corresponds to image data which would have been reconstructed on the basis of projection measurement data which would have been generated by the proportion of X-rays of the total spectrum of X-rays detected which only have radiation with the respective underlying energy value. The calculation of the monoenergetic image data, in particular the first and second monoenergetic image data, comprises a reconstruction or image data reconstruction on the basis of the respectively assigned projection measurement data.

As a rule, the first and second attenuation values differ due to the strong irrigation with contrast agent in the area of the heart during diastole, when the heart is expanded, whereas at the time of systole, when the heart is contracted, the heart is relatively anemic and thus also poor in contrast agent and the contrasts are thus also significantly less pronounced.

In particular, it may be provided that an alignment takes place of the attenuation values and thus also of the contrasts of the image data which are to be assigned to diastole, and of the image data which are to be assigned to systole. In this way, in particular the contrasts in the image data associated with systole are greatly improved. Furthermore, by aligning the contrasts of the image data obtained at different times and during different states of the heart, the dynamic behavior of the heart can be examined without having to perform additional scans, which would entail additional radiation exposure.

A second energy value is therefore determined on the basis of the first attenuation value and/or the second attenuation value, for which third monoenergetic image data is reconstructed on the basis of the first spectrally differentiated CT projection measurement data. As the first monoenergetic image data, the assigned first projection measurement data of which was acquired during diastole, has very high attenuation values compared to the second monoenergetic image data, the second energy value is now selected in such a way that these attenuation values are somewhat reduced. This objective is achieved when using conventional contrast agents, such as for example iodine, by selecting a higher second energy value than the first energy value, both energy values generally being above the energy value of the X-ray threshold of the contrast agent, preferably iodine. In this case, the second energy value, in particular in the case of the contrast agent iodine, is further away from the X-ray threshold or absorption edge assigned to the contrast agent than the first energy value, so that the effect of the contrast agent is reduced.

Based on the first attenuation value and/or the second attenuation value, a third energy value is also determined, for which fourth monoenergetic image data is reconstructed on the basis of the second spectrally differentiated CT projection measurement data. As the second monoenergetic image data, the assigned second projection measurement data of which was acquired during systole, has very low attenuation values compared to the first monoenergetic image data, the third energy value is now selected in such a way that these attenuation values in the fourth monoenergetic image data increase somewhat compared to the attenuation values of the second monoenergetic image data. This objective is achieved by selecting the third energy value to be lower than the first energy value. In this case, the third energy value is closer to the absorption edge assigned to the contrast agent than the first energy value, for example, by 33 keV in the case of iodine as a contrast agent, so that the effect of the contrast agent is enhanced.

The second energy value and the third energy value can therefore be determined in such a way that an alignment of an attenuation caused by the contrast agent in the third monoenergetic image data and the fourth monoenergetic image data is compared to an attenuation caused by the contrast agent in the first monoenergetic image data and the second monoenergetic image data.

Finally, the third monoenergetic image data for the determined second energy value is calculated on the basis of the first spectrally differentiated CT projection measurement data and the fourth monoenergetic image data for the determined third energy value is calculated on the basis of the second spectrally differentiated CT projection measurement data. The calculation of the third monoenergetic image data and fourth monoenergetic image data comprises, in particular, a reconstruction of the third monoenergetic image data and fourth monoenergetic image data.

The second energy value can be determined in particular based on the first monoenergetic image data and/or the second monoenergetic image data. The third energy value can be determined in particular based on the first monoenergetic image data and/or the second monoenergetic image data.

The attenuation caused by the contrast agent may in particular relate to a representative sub-area of the heart. The alignment of the attenuation caused by the contrast agent in the third monoenergetic image data and the attenuation caused by the contrast agent in the fourth monoenergetic image data with one another can be performed in particular in comparison to a difference between an attenuation caused by the contrast agent in the first monoenergetic image data and an attenuation caused by the contrast agent in the second monoenergetic image data.

The alignment of the attenuation caused by the contrast agent in the third monoenergetic image data and the attenuation caused by the contrast agent in the fourth monoenergetic image data with one another can be performed in particular in such a way that a difference between an attenuation caused by the contrast agent in the third monoenergetic image data and an attenuation caused by the contrast agent in the fourth monoenergetic image data is smaller than a difference between an attenuation caused by the contrast agent in the first monoenergetic image data and an attenuation caused by the contrast agent in the second monoenergetic image data.

An alignment therefore takes place of the attenuation values of the image data recorded at different times and for different cardiac conditions. This objective is achieved because the recording of spectrally differentiated image data enables an alignment of the contrasts through a targeted selection of energy values of monoenergetic image data. In this way, it is possible to align contrasts of image data which are characterized by a very different intrinsic contrast due to the different time of the respective acquisition of the projection measurement data assigned to them and the associated natural physiological timing of the cardiac function. In this way, the items of monoenergetic image data obtained can be offset against each other in suitable further processing without the influence of systematic errors due to the difference in contrast.

By achieving a comparable contrast in the various reconstructions, a conventional threshold-based segmentation of the chambers of the heart and the myocardium is possible without systematic errors. As part of such an evaluation, the stroke volume of the main chamber of the left ventricle is subsequently determined and the relative change and thus the ejection fraction is determined from the measurements in the systole and diastole, i.e. the proportion of blood which the heart ejects during a heartbeat in relation to the total volume of the heart).

This procedure makes it possible to obtain additional CT-based diagnostic information, in particular with regard to the myocardial wall thickness and the dynamic behavior of the myocardium during the transition between diastole and systole, without having to use an acquisition with increased radiation exposure as is conventional. Advantageously, the contrast between the myocardium and the ventricles, in particular the left ventricle, of the image data generated during systole is significantly improved, so that statements can be made about the wall thickness and the dynamic behavior of the myocardium during cardiac activity. Furthermore, the contrasts in the systolic image data processed in this way are sufficient to be able to make a statement regarding the extracellular volume of a heart and the fibrotic changes in the heart which may be associated with it. In particular, a cardiac deformation analysis can be carried out particularly precisely, with which damage to the heart, such as that which can occur during chemotherapy, for example, can be detected at an early stage and counteracted early on. It should be explicitly summarized again at this point that the first monoenergetic image data is preferably used for an angiography examination in order to examine the vessels of the heart. A combination of the third and fourth monoenergetic image data is preferably used for the examination of the dynamic behavior of the heart and the fourth monoenergetic image data is used for the ECV examination. Advantageously, due to the multiple use of the image data for different types of examination, the total time of imaging can be reduced, which also reduces the radiation dose for the patient.

The image generating facility, according to an embodiment of the present invention, has an input interface which is configured to receive first spectrally differentiated CT projection measurement data. The first spectrally differentiated CT projection measurement data relates to a heart of a patient in a first time interval, a contrast agent mainly being located in the chambers and the vessels of the heart during the first time interval, the first time interval comprising a diastole of the heart. The input interface is furthermore configured to receive second spectrally differentiated CT projection measurement data. The second spectrally differentiated CT projection measurement data relates to the heart of the patient in a later second time interval, the contrast agent mainly being located in the muscle tissue of the heart during the second time interval, the second time interval comprising a systole of the heart.

Part of the image generating facility, according to an embodiment of the present invention, is also a reconstruction unit which is configured to calculate first monoenergetic image data based on the first spectrally differentiated CT projection measurement data for a first energy value. The reconstruction unit is also configured to calculate second monoenergetic image data based on the second spectrally differentiated CT projection measurement data for the first energy value.

The image generating facility, according to an embodiment of the present invention, also comprises an alignment unit for determining a second energy value for which third monoenergetic image data is reconstructed, and a third energy value for which fourth monoenergetic image data is reconstructed, such that an alignment of an attenuation caused by the contrast agent in the third monoenergetic image data and the fourth monoenergetic image data is performed in comparison to an attenuation caused by the contrast agent in the first monoenergetic image data and the second monoenergetic image data.

The reconstruction unit of the image generating facility, according to an embodiment of the present invention, is furthermore configured to calculate the third monoenergetic image data for the determined second energy value on the basis of the first spectrally differentiated CT projection measurement data and to calculate the fourth monoenergetic image data for the determined third energy value on the basis of the second spectrally differentiated CT projection measurement data.

The image generating facility, according to an embodiment of the present invention, shares the advantages of the method, according to an embodiment of the present invention, for spectrally differentiated cardiac CT imaging.

The computed tomography system, according to an embodiment of the present invention, has a scanning unit with a photon-counting X-ray detector and a control facility (or control device) for controlling the scanning unit and for evaluating the raw data generated by the scanning unit or projection measurement data. The control facility also includes an image generating facility according to the invention. The computed tomography system, according to an embodiment of the present invention, shares the advantages of the image generating facility according to the invention.

A majority of the aforementioned components of the image data generating facility (or image data generating device), according to an embodiment of the present invention, can be realized in whole or in part in the form of software modules in a processor of a corresponding computing system, for example by a control facility of a computed tomography system or a computer which is used to control such a system. A largely software-based implementation has the advantage that previously used computing systems can also be easily retrofitted via a software update in order to work in the manner according to the invention. In this respect, the object is also achieved by a corresponding computer program product with a computer program which can be loaded directly into a computing system, with program sections to carry out the steps of the method, according to an embodiment of the present invention, for spectrally differentiated cardiac CT imaging when the program is executed in the computing system. In addition to the computer program, such a computer program product may include additional components such as, for example, documentation and/or additional components, including hardware components such as, for example, hardware keys (dongles, etc.) for use of the software.

A non-transitory computer-readable medium, e.g. a memory stick, a hard disk or another portable or permanently installed data carrier, on which the program sections of the computer program which can be imported and executed by a computing system are stored, can be used for transport to the computing system or the control facility and/or for storage on or in the computing system or the control facility. The computing system may, for example, have one or more cooperating microprocessors or the like for this purpose.

The dependent claims and the following description each contain particularly advantageous embodiments and developments of the invention. In particular, the claims of one category of claims can also be developed analogously to the dependent claims of another category of claims. In addition, within the scope of the invention, the various features of different exemplary embodiments and claims can also be combined to form new exemplary embodiments.

In a preferred embodiment of the method, according to the present invention, for spectrally differentiated cardiac CT imaging, the step of determining a second energy value and a third energy value comprises the following partial steps:

First, a first attenuation value is determined in a representative sub-area of the first monoenergetic image data. The term “representative sub-area” is to be understood as a sub-area of the examination area, i.e. in particular of the heart, which contains as much contrast agent as possible during the acquisition of the projection measurement data. Advantageous sub-areas preferably include the aorta or the left ventricle, in which a particularly large amount of blood flows or is present. The first attenuation value is preferably determined as an average value of an attenuation in the representative sub-area in the first monoenergetic image data. Alternatively, the first attenuation value can also be determined as a maximum value or a minimum value of an attenuation in the representative sub-area or as a value at a predetermined position in the representative sub-area.

Preferably, the representative sub-area is selected in such a way that the largest possible homogeneous area of iodine contrast agent is obtained and in particular, the large vessel of the aorta or the left ventricle of the heart have proven to be anatomically advantageous for measurement.

Furthermore, a second value of an attenuation caused by the contrast agent in the representative sub-area is also determined in the second monoenergetic image data. The second value is preferably determined as an average value of an attenuation in the representative sub-area in the second monoenergetic image data. Alternatively, the second value can also be determined as a value at the same position in the representative sub-area of the second monoenergetic image data as in the representative sub-area of the first monoenergetic image data. Alternatively again, the second value can also be determined as the maximum value or minimum value of an attenuation in the representative sub-area or as a value at a predetermined position in the representative sub-area of the second monoenergetic image data.

In particular, the second energy value can be determined on the basis of the first attenuation value and the third energy value can be determined on the basis of the second attenuation value. The second energy value and the third energy value are determined in such a way that a difference between an attenuation caused by the contrast agent in the representative sub-area in the third monoenergetic image data and the fourth monoenergetic image data is reduced in comparison to the difference between the first attenuation value and the second attenuation value. The attenuation caused by the contrast agent in the representative sub-area in the third monoenergetic image data and the fourth monoenergetic image data is determined analogously to the determination of the first attenuation value and the second attenuation value. Preferably, the determination of the attenuation caused by the contrast agent in the representative sub-area in the third monoenergetic image data and the fourth monoenergetic image data is carried out using exactly the same method as the determination of the first attenuation value and the second attenuation value. Advantageously, reference variables on the basis of which the alignment of the contrasts in the third and fourth monoenergetic image data is carried out can be particularly precise and representative.

In a preferred variant of the method, according to an embodiment of the present invention, for spectrally differentiated cardiac CT imaging, the second energy value and the third energy value are determined in such a way that a difference between an attenuation caused by the contrast agent in the third monoenergetic image data and an attenuation caused by the contrast agent in the fourth monoenergetic image data is minimized. Advantageously, in this variant, the image data obtained during diastole and the image data obtained during systole, are aligned as closely as possible with regard to their contrast or their attenuation values. Advantageously, wall structures can be made particularly clear during systole of the heart as in particular the contrast in the fourth monoenergetic image data is increased by the alignment. Furthermore, the third and fourth monoenergetic image data can be offset against one another particularly well due to maximum alignment in order to be able to display the dynamic behavior of the heart muscle particularly realistically.

When aligning the contrasts of the first and second monoenergetic image data, the second energy value and the third energy value are preferably selected on the basis of a physics table which specifies a functional relationship between a concentration of a contrast agent, an energy value and an attenuation value. Specifically for the material, this alignment would be based, for example, on the mass attenuation coefficient for X-rays. Such data is available, for example, from the National Institute of Standards and Technology. Advantageously, the second and third energy values to be selected for the alignment of the contrasts can simply be selected from a database without complex calculations having to be performed again.

The contrast agent used for contrast imaging is particularly preferably iodine. Iodine is particularly suitable for visualizing blood, which first makes the vessels and ventricles visible when the heart is irrigated and then diffuses into the heart muscle at a later stage of irrigation, after approximately 3 to 5 minutes, and can be used to contrast the heart walls when using the method according to the invention.

In the method, according to an embodiment of the present invention, for spectrally differentiated cardiac CT imaging, spectrally differentiated CT projection measurement data is preferably recorded as spectrally resolved CT projection measurement data. Advantageously, such projection measurement data is particularly suitable for calculating monoenergetic image data with different energy values due to its spectral information.

Such spectrally resolved CT projection measurement data is particularly preferably recorded by a photon-counting X-ray detector. With such a photon-counting X-ray detector, X-rays can be detected broken down into individual spectral components. This spectrally decomposed projection measurement data is particularly suitable for generating monoenergetic image data.

When reconstructing the third monoenergetic image data and the fourth monoenergetic image data, the following reconstruction parameters are preferably synchronized and their values are selected identically:
the layer thickness of the image data,
a dimension of a reconstruction kernel for the filtered back projection,
the dimension of a field of view.

Advantageously, items of image data recorded at different times can be easily combined due to their identical structure or their identical dimensions.

Reconstruction of the third monoenergetic image data and the fourth monoenergetic image data preferably takes place directly in the CT system. Advantageously, all other parameters which would possibly lead to a systematic deviation in the different image data are also synchronized as part of the reconstruction.

Alternatively, the reconstruction of the third monoenergetic image data and the fourth monoenergetic image data first takes place with different reconstruction parameter values. As part of post-processing, common image parameter values of the monoenergetic image data are formed on the basis of the maximum and/or minimum of the reconstruction parameter values of the third monoenergetic image data and/or the fourth monoenergetic image data. For this purpose, there should be access to a spectrally resolved data format, for example SPP, which offers the possibility of generating images with different energy levels (keV level). The image parameter values can be harmonized on the basis of extreme values of the different reconstruction parameter values as part of post-processing in order to be able to combine the third and fourth monoenergetic image data without distortion.

The reconstruction parameters aligned in this way preferably comprise a layer thickness and/or a dimension of a reconstruction kernel with which the reconstruction of the image data was performed. While the maximum (for example, max (0.4 mm, 1 mm)) is preferably used as the aligned reconstruction parameter of the different image data for the layer thicknesses, the minimum (for example, min (Bv44, Qr36)) is preferably used when aligning the dimensions of the reconstruction kernel (Bv stands for “Body vascular”, Qr stands for “Quantitative regular”, i.e. kernels for different body areas and components of the body). In this way, the image dimensions and the image sharpness of the different items of image data are aligned with one another. Filtering is used to make the “sharper” image “less sharp” or “softer”. The number assigned to a kernel reflects the spatial resolution achieved in the image and has a one-to-one relationship to the modulation transfer function value at 60 percent of this kernel. The modulation transfer function value provides information regarding the relationship between the detail contrast at the edges of an object and the detail contrast of its visual representation. With minimization, when comparing two images of different sharpness, the sharper image is “softened” by filtering, so that the sharper image, which was reconstructed with the Bv44 kernel, is aligned with an image which is softer or less sharp and which is reconstructed with the Qr36 kernel.

The common image parameter values of the post-processed third and fourth monoenergetic image data are preferably determined by the use of a low-pass filter in the z-direction or in the in-plane direction to the first and/or second monoenergetic image data and/or to the third and/or fourth monoenergetic image data. With the low-pass filter, the image textures can be aligned with one another as higher-frequency image changes are suppressed. As usual, the z-direction should be understood as the axis of rotational symmetry of a computed tomography system. Layers are usually aligned transversely to the longitudinal axis of the patient.

In a preferred embodiment of the image generating facility according to the present invention, the image generating facility, according to an embodiment of the present invention, comprises an image analysis unit for determining a first attenuation value in a representative sub-area of the first monoenergetic image data and for determining a second attenuation value in the same representative sub-area in the second monoenergetic image data.

In this embodiment, the alignment unit is configured to determine the second energy value on the basis of the attenuation caused by the contrast agent in a representative sub-area of the first monoenergetic image data and to determine the third energy value on the basis of the attenuation caused by the contrast agent in a representative sub-area of the second monoenergetic image data. The alignment unit is configured so that the second energy value and the third energy value are selected in such a way that a difference between an attenuation caused by the contrast agent in the representative sub-area in the third monoenergetic image data and the fourth monoenergetic image data is reduced in comparison to the difference between the first attenuation value and the second attenuation value. The representative sub-area reflects the differences in contrast in heart formation between diastole and systole in a particularly pronounced manner, so that a particularly precise alignment of the contrasts in the third and fourth monoenergetic image data is achieved if a representative sub-area is selected appropriately.

### BRIEF DESCRIPTION OF THE DRAWINGS

Individual features of the present invention are explained hereinafter with reference to the attached figures by examples. The figures show:

FIG. 1A diagrammatic view of a computed tomography system,

FIG. 2A flow chart which illustrates a method for spectrally differentiated heart CT imaging,

FIG. 3A diagrammatic sectional view of an image generating facility,

FIG. 4A contrast agent-enhanced image display of the heart at the diastole stage with contrast agent in the left ventricle and the blood vessels,

FIG. 5A contrast agent-enhanced image display of the heart at the systole stage with contrast agent in the muscle tissue,

FIG. 6A contrast agent-enhanced image display of the heart at the diastole stage with contrast agent in the left ventricle and the blood vessels, aligned by the method for spectrally differentiated heart CT imaging

FIG. 7A contrast agent-enhanced image display of the heart at the systole stage with contrast agent in the muscle tissue, aligned by the method for spectrally differentiated heart CT imaging.

### DETAILED DESCRIPTION

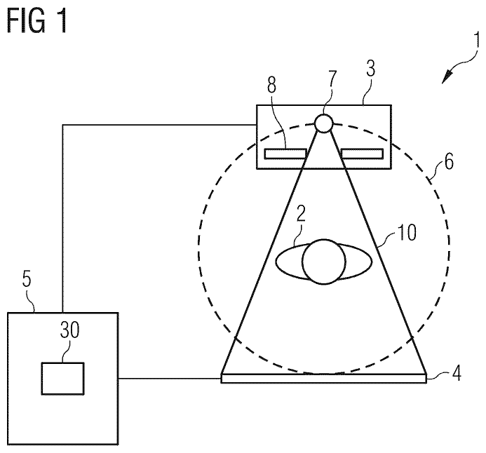

FIG. 1 shows a diagrammatic view of a computed tomography system 1. The computed tomography system 1 shown in FIG. 1 here comprises an X-ray emitter arrangement with an X-ray radiation source 3, an X-ray detector 4 and a control facility 5 (also referred to as a control device 5). The X-ray radiation source 3 and the X-ray detector 4 are connected to the control facility 5. The X-ray radiation source 3 and the X-ray detector 4 are movable and arranged diametrically to one another on a circular path 6. They are therefore in a fixed position in relation to one another, in which the X-ray detector 4 detects the radiation emitted by the X-ray radiation source 3 and thus form a first source X-ray detector arrangement. A patient 2 is located in the center of the circular path 6 as the object for examination. The X-ray radiation source 3 comprises an X-ray tube 7 and an aperture 8. The aperture 8 is arranged at a slight distance from the X-ray tube 7 on a side of the X-ray tube 7 facing the patient 2. It can be used to adjust an exit angle of X-rays 10 emitted by the X-ray tube 7 during operation.

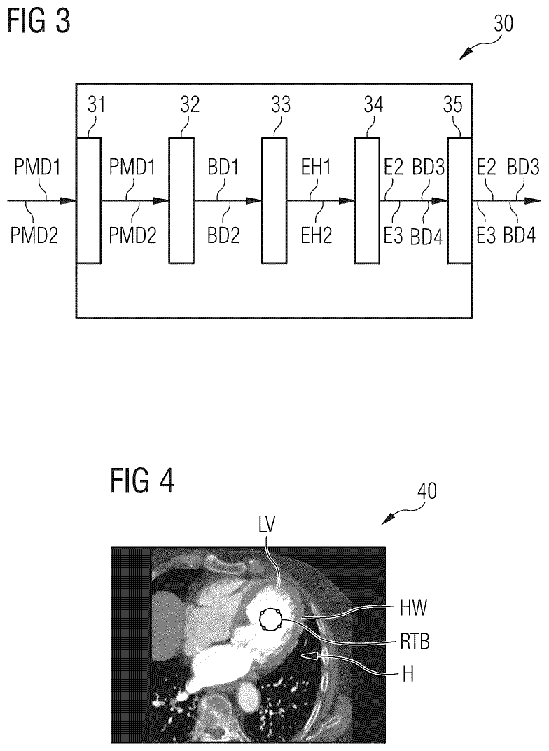

During operation, the X-ray radiation source 3 and the X-ray detector 4 are rotated around the patient 2 on the circular path 6 to acquire projection measurement data. The acquired projection measurement data can then be transmitted to an evaluation unit located, for example, in the control facility 5 and reconstructed there into an image of the patient 2. In order to acquire projection measurement data from other areas of the patient 2, the patient 2 can be moved relative to the computed tomography system 1, for example via a positionable patient table (not shown here), perpendicular to the plane of the circular path 6. In the case of so-called spiral CT, acquisition takes place continuously with a likewise continuous table feed. The recorded projection measurement data is then divided into its spectral components for reconstruction and a reconstruction based on the spectral resolution is performed. The reconstruction takes place in a control facility or control device (not shown), which is, for example, either directly part of the computed tomography system 1 or is configured as software in a downstream computer. In particular, the control facility 5 comprises an image generating facility 30 (also referred to as an image generating device 30) shown in detail in FIG. 3. The image data generated by the image generating facility 30 can be further processed in a computer (not shown) which is electrically connected to the computed tomography system 1 and displayed on a computer screen (not shown). A contrast agent can also be injected into the heart area of the patient via an injection facility or injection device (not shown) in order to be able to visualize both vascular areas and the heart muscle itself at intervals of approximately 3 to 5 minutes.

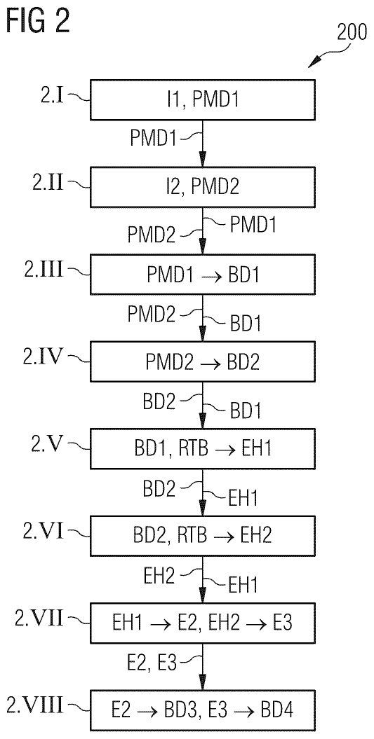

FIG. 2 shows a flow chart which illustrates a method for spectrally differentiated cardiac CT imaging.

In step 2.I, after irrigating the heart region and in particular the ventricles and vessels of the heart of a patient with a contrast agent, in particular iodine, contrast agent-enhanced first spectrally resolved CT projection measurement data PMD1 of the heart of the patient is acquired in a first time interval I1. In the first time interval I1, the contrast agent is mainly located in the ventricles and the vessels of the heart and the first time interval I1 comprises a diastole of the heart. I.e., the heart is relaxed to the maximum and the ventricles are filled with blood.

In step 2.II, approximately 3 to 5 minutes after step 2.I, second spectrally resolved CT projection measurement data PMD2 is acquired from the heart of a patient in a later second time interval I2, during which the contrast agent is mainly located in the muscle tissue of the heart. The second time interval I2 comprises a systole of the heart.

In step 2.III, first monoenergetic image data BD1 is calculated on the basis of the first spectrally differentiated CT projection measurement data PMD1 for a first energy value E1. This first energy value is selected in such a way that the brightness of the areas with contrast agent in the first monoenergetic image data BD1 is particularly high, so that this image data BD1 is particularly suitable for angiographic imaging.

Furthermore, in step 2.IV, second monoenergetic image data BD2 is reconstructed or calculated on the basis of the second spectrally differentiated CT projection measurement data PMD2 for the first energy value E1. As a rule, the contrast in the second monoenergetic image data BD2 is now much weaker than in the first monoenergetic image data BD1 as at the time of acquisition of the second projection measurement data BD2 only relatively little contrast agent still remains in the heart. In particular, the contrast in the second monoenergetic image data BD2 is usually so weak that the wall structures of the heart cannot be recognized with sufficiently reliability and precision or cannot be sufficiently distinguished from the area of the ventricles in the second monoenergetic image data BD2.

In order to align the contrasts in the first monoenergetic image data BD1 and the second monoenergetic image data BD2, in step 2.V a first attenuation value EH1 is first determined in a representative sub-area RTB of the first monoenergetic image data BD1. A central area of the left ventricle, which has particularly high brightness values or attenuation values during diastole, can be used as a representative sub-area RTB in the first monoenergetic image data BD1.

Furthermore, in step 2.VI, a second attenuation value EH2 is determined in the second monoenergetic image data BD2 in the representative sub-area RTB, i.e. at the same point at which the first attenuation value EH1 of an attenuation caused by the contrast agent was determined in the first monoenergetic image data BD1.

As a rule, the second attenuation value EH2 is significantly lower than the first attenuation value EH1 as the contrast agent has already largely moved out of the heart during the second time interval I2. Only the muscle tissue still contains contrast agent, but this has a much lower concentration than the amount of contrast agent which was present in the ventricles during the first time interval I1.

In order to now achieve an alignment of the contrasts of the image data BD1, BD2 recorded at different times, in step 2.VII a second energy value E2 is determined on the basis of the first attenuation value EH1 and third monoenergetic image data BD3 based on the first spectrally differentiated CT projection measurement data PMD1 is reconstructed for the second energy value E2.

In step 2.VIII, a third energy value E3 is determined on the basis of the second attenuation value EH2 and fourth monoenergetic image data BD4 is reconstructed for the third energy value E3 on the basis of the second spectrally differentiated CT projection measurement data PMD2. The second energy value E2 and the third energy value E3 are selected in such a way that a difference between an attenuation caused by the contrast agent in the respective representative sub-areas of the third and fourth monoenergetic image data BD3, BD4 is reduced.

FIG. 3 shows a diagrammatic view of an image generating facility 30.

The image generating facility 30 has an input interface 31. The input interface 31 is configured to receive first spectrally resolved CT projection measurement data PMD1. The spectrally resolved CT projection measurement data PMD1 was acquired from the heart of a patient in a first time interval I1 with the aid of a contrast agent. This first time interval I1 was selected in such a way that during the first time interval I1 the contrast agent is mainly located in the ventricles and the vessels of the heart and that the first time interval I1 comprises a diastole of the heart.

The input interface 31 is also configured to receive second spectrally differentiated CT projection measurement data PMD2. The second spectrally differentiated CT projection measurement data PMD2 relates to the heart of the patient at a later second time interval I2, the contrast agent mainly being located in the muscle tissue of the heart during the second time interval I2. As a rule, the second time interval I2 is selected in such a way that it is 3 to 5 minutes after the first time interval. The second time interval I2 comprises a systole of the heart. During systole, there is only a little blood and therefore also little contrast agent in the ventricles, so that a visual reproduction of the heart can be expected to have reduced contrast at this time.

Part of the image generating facility 30 is also a reconstruction unit 32 which is configured to calculate first monoenergetic image data BD1 based on the first spectrally differentiated CT projection measurement data PMD1 for a first energy value E1 and to calculate second monoenergetic image data BD2 based on the second spectrally differentiated CT projection measurement data for the first energy value E1.

The image generating facility 30 also has an image analysis unit 33 which is configured to determine a first attenuation value EH1 in a representative sub-area RTB of the first monoenergetic image data BD1 and to determine a second attenuation value EH2 in the representative sub-area RTB in the second monoenergetic image data BD2. The representative sub-area RTB is selected in such a way that a particularly large amount of contrast agent is present there during the diastolic phase in order to obtain the most meaningful difference possible between the first attenuation value EH1 and the second attenuation value EH2.

As a rule, the representative sub-area RTB is selected in such a way that the largest possible homogeneous area of iodine contrast agent is obtained and the large vessel of the aorta or the left ventricle of the heart have proven to be anatomically advantageous for a measurement.

The image generating facility 30 also has an alignment unit 34. The alignment unit 34 is configured to determine a second energy value E2 and a third energy value E3 on the basis of the first attenuation value EH1 and the second attenuation value EH2. Third monoenergetic image data BD3 based on the first spectrally differentiated CT projection measurement data PMD1 is reconstructed for the second energy value E2. Fourth monoenergetic image data BD4 based on the second spectrally differentiated CT projection measurement data PMD2 is reconstructed for the third energy value E3. The second energy value E2 and the third energy value E3 are selected in such a way that a difference between an attenuation caused by the contrast agent in the respective representative sub-areas of the third and fourth monoenergetic image data BD3, BD4 is reduced. Part of the image generating facility 30 shown in FIG. 3 is also an output interface 35, with which the third and fourth monoenergetic image data BD3, BD4 aligned regarding their contrast behavior are output.

FIG. 4 shows a contrast agent-enhanced image display 40 of the heart H at the diastole stage with contrast agent in the left ventricle LV and the blood vessels. A circular representative sub-area RTB is shown in the left ventricle LV, in which an average attenuation value is determined. Furthermore, in the image display 40 shown in FIG. 4, the heart wall HW, which is darkly contrasted with the light inner area of the left ventricle LV, can be clearly seen. At this early stage after irrigation with a contrast agent, the contrast agent is still located in the ventricles and the vessels, which are shown as light in color in FIG. 4. The actual heart muscle or the heart wall, on the other hand, is not yet penetrated by the contrast agent and is therefore shown as dark in color in FIG. 4. In the nomenclature already used above, first monoenergetic image data BD1 reconstructed with a first energy value E1 are shown in FIG. 4. Such a first energy value E1 can be, for example, 70 keV.

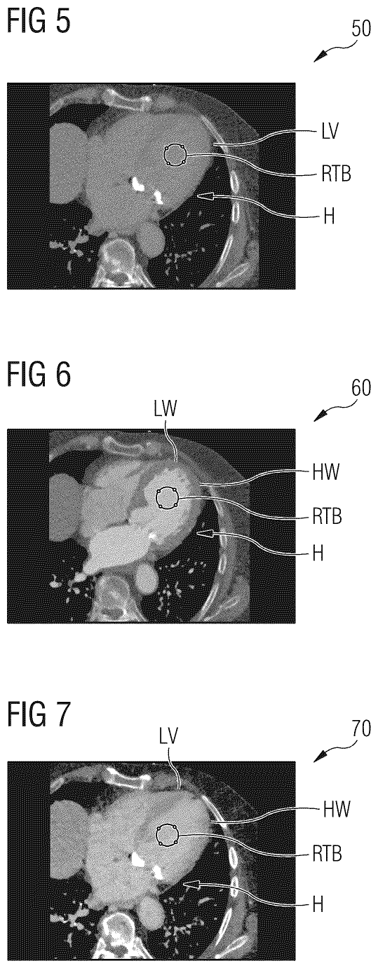

FIG. 5 shows a contrast agent-enhanced image display 50 of the heart H at the stage of a systole with contrast agent in the muscle tissue. At the stage shown in FIG. 5, the left ventricle LV is shown in gray as the contrast agent has been distributed in the muscle at this stage. The circular representative sub-area RTB is now also colored dark gray. The heart walls are no longer visible in FIG. 5 as they are no longer distinct from the left ventricle LV. In the nomenclature already used above, in FIG. 5 second monoenergetic image data BD2 is shown reconstructed with the first energy value E1 which, as already mentioned, can be, for example, 70 keV.

In order to make the image displays of the diastole and the systole comparable and, in particular, to differentiate the heart walls from the left ventricle LV in the representation during systole, the energy values E2, E3 for the generation of third and fourth monoenergetic image data BD3, BD4 should now be selected in such a way that the contrasts in the diastolic image display and the systolic image display are comparable. For this purpose, the energy values E2, E3 must be selected significantly differently as the contrast agent concentrations vary greatly between diastole and systole.

FIG. 6 shows a contrast agent-enhanced image display 60 of the heart at the diastole stage with contrast agent in the left ventricle and the blood vessels, aligned by the method. FIG. 6 therefore shows third monoenergetic image data BD3 reconstructed for a second energy value E2. For example, 85 keV can be used as a second energy value. This means that the second energy value is further away from the iodine absorption edge than at a value of 70 keV, which attenuates the contrasts caused by the contrast agent. As can be seen in FIG. 6, the contrasts of the image data referred to as third monoenergetic image data BD3 in FIG. 6 in the above nomenclature are actually somewhat attenuated compared to the first image data BD1 shown in FIG. 4, but still strong enough for the heart walls HW to be sufficiently differentiated from the left ventricle LV of the heart H. The circular representative sub-area RTB shown in FIG. 6 is now no longer colored white, but gray.

FIG. 7 shows a contrast agent-enhanced image display 70 of the heart H at the systole stage with contrast agent in the muscle tissue, aligned by the method. FIG. 7 therefore shows image data referred to as fourth monoenergetic image data BD3 in the nomenclature used above, which was reconstructed with a third energy value E3. For example, a value of 45 keV can be used as the third energy value E3. This value is sufficiently close to the absorption edge of iodine, so that the contrast in FIG. 7 is considerably enhanced compared to FIG. 5, in which the energy value E1 was 70 keV. As a result of the contrasts being significantly enhanced compared to the second monoenergetic image data BD2 shown in FIG. 5, the heart walls HW are differentiated sufficiently strongly from the left ventricle LV of the heart H to be able to segment the heart walls. The circular representative sub-area RTB shown in FIG. 7 is now no longer colored dark gray but light gray and is differentiated at least somewhat from the heart walls HW. Furthermore, the contrasts in FIG. 6 and FIG. 7 are aligned with one another compared to FIG. 4 and FIG. 5.

It is pointed out once again that the method and apparatuses described above are merely preferred embodiments and that the present invention can be varied by a person skilled in the art without departing from the scope of the invention, insofar as defined by the claims. For the sake of completeness, it is also pointed out that the use of the indefinite article “a” or “an” does not exclude the possibility that the features concerned may also be present more than once. Likewise, the term “unit” does not exclude the possibility that it may consist of a plurality of components which may also be spatially distributed.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility, data processing device or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for spectrally differentiated cardiac CT imaging, the computer-implemented method comprising:
receiving, in a first time interval, first spectrally differentiated CT projection measurement data that relates to a heart of a patient, wherein a contrast agent is located in at least one of chambers or vessels of the heart during the first time interval, and wherein the first time interval includes a diastole of the heart;
receiving, in a second time interval, second spectrally differentiated CT projection measurement data THAT relates to the heart of the patient, wherein the contrast agent is located in a muscle tissue of the heart during the second time interval, and wherein the second time interval includes a systole of the heart;
calculating first monoenergetic image data for a first energy value based on the first spectrally differentiated CT projection measurement data;
calculating second monoenergetic image data for the first energy value based on the second spectrally differentiated CT projection measurement data;
determining a second energy value and a third energy value;
calculating third monoenergetic image data for the second energy value based on the first spectrally differentiated CT projection measurement data; and
calculating fourth monoenergetic image data for the third energy value based on the second spectrally differentiated CT projection measurement data; wherein
the second energy value and the third energy value are determined such that an attenuation caused by the contrast agent in the third monoenergetic image data is aligned with an attenuation caused by the contrast agent in the fourth monoenergetic image data.

2. The computer-implemented method as claimed in claim 1, wherein the determining a second energy value and a third energy value comprises:
determining a first attenuation value that relates to an attenuation caused by the contrast agent in the first monoenergetic image data;
determining a second attenuation value that relates to an attenuation caused by the contrast agent in the second monoenergetic image data;
determining the second energy value based on at least one of the first attenuation value or the second attenuation value; and
determining the third energy value based on at least one of the first attenuation value or the second attenuation value; wherein
the second energy value and the third energy value are determined such that a difference between the attenuation caused by the contrast agent in the third monoenergetic image data and the attenuation caused by the contrast agent in the fourth monoenergetic image data is reduced compared to a difference between the first attenuation value and the second attenuation value.

3. The computer-implemented method as claimed in claim 1, wherein the second energy value and the third energy value are determined such that a difference between the attenuation caused by the contrast agent in the third monoenergetic image data and the attenuation caused by the contrast agent in the fourth monoenergetic image data is minimized.

4. The computer-implemented method as claimed in claim 1, wherein the second energy value and the third energy value are determined based on a physics table that indicates a functional relationship between energy values and attenuation values.

5. The computer-implemented method as claimed in claim 1, wherein the first spectrally differentiated CT projection measurement data and the second spectrally differentiated CT projection measurement data are spectrally resolved CT projection measurement data.

6. The computer-implemented method as claimed in claim 5, wherein the spectrally resolved CT projection measurement data is recorded by a photon-counting X-ray detector.

7. The computer-implemented method as claimed in claim 1, wherein during the calculating of the third monoenergetic image data and during the calculating of the fourth monoenergetic image data, the following reconstruction parameters are at least one of synchronized or have values selected identically:
a layer thickness of image data,
a dimension of a reconstruction kernel for a filtered back projection, and
a dimension of a field of view.

8. The computer-implemented method as claimed in claim 7, wherein the calculating of the third monoenergetic image data and the calculating of the fourth monoenergetic image data are performed directly in a computed tomography system used to acquire the first spectrally differentiated CT projection measurement data and the second spectrally differentiated CT projection measurement data.

9. The computer-implemented method as claimed in claim 1, wherein in the context of post-editing of the third monoenergetic image data and the fourth monoenergetic image data, common image parameter values for post-edited third monoenergetic image data and post-edited fourth monoenergetic image data are determined based on at least one of a maximum or a minimum of values of reconstruction parameters of at least one of the third monoenergetic image data or the fourth monoenergetic image data.

10. The computer-implemented method as claimed in claim 9, wherein the reconstruction parameters include at least one of a layer thickness or a dimension of a reconstruction kernel.

11. The computer-implemented method as claimed in claim 9, wherein the common image parameter values are determined by applying a low-pass filter in a z-direction or in a layer direction as part of the post-editing of the third monoenergetic image data and the fourth monoenergetic image data.

12. An image generating device, comprising:
an input interface configured to
receive, in a first time interval, first spectrally differentiated CT projection measurement data that relates to a heart of a patient, wherein a contrast agent is located in at least one of chambers or vessels of the heart during the first time interval, and wherein the first time interval includes a diastole of the heart, and
receive, in a second time interval, second spectrally differentiated CT projection measurement data that relates to the heart of the patient, wherein the contrast agent is located in a muscle tissue of the heart during the second time interval, and wherein the second time interval includes a systole of the heart,
a reconstruction unit configured to
calculate first monoenergetic image data for a first energy value based on the first spectrally differentiated CT projection measurement data,
calculate second monoenergetic image data for the first energy value based on the second spectrally differentiated CT projection measurement data,
calculate third monoenergetic image data for a second energy value based on the first spectrally differentiated CT projection measurement data, and
calculate fourth monoenergetic image data for a third energy value based on the second spectrally differentiated CT projection measurement data; and
an alignment unit configured to determine the second energy value and the third energy value such that an attenuation caused by the contrast agent in the third monoenergetic image data is aligned with an attenuation caused by the contrast agent in the fourth monoenergetic image data.

13. A computed tomography system, comprising:
a scanning unit with a photon-counting X-ray detector, and
a control device configured to
control the scanning unit, and
receive projection measurement data from the scanning unit, wherein the control device includes the image generating device as claimed in claim 12.

14. A non-transitory computer program product comprising commands that, when executed by a computer, cause the computer to carry out the computer-implemented method as claimed in claim 1.

15. A non-transitory computer-readable storage medium storing computer-executable instructions that, when executed by at least one processor at an image generating device, cause the image generating device to carry out the computer-implemented method as claimed in claim 1.

16. The computer-implemented method as claimed in claim 1, wherein the contrast agent is iodine.

17. The computer-implemented method as claimed in claim 4, wherein the second energy value and the third energy value are determined as a function of a concentration of the contrast agent.

18. The image generating device of claim 12, wherein the contrast agent is iodine.

19. The computer-implemented method as claimed in claim 2, wherein in the context of post-editing of the third monoenergetic image data and the fourth monoenergetic image data, common image parameter values for post-edited third monoenergetic image data and post-edited fourth monoenergetic image data are determined based on at least one of a maximum or a minimum of values of reconstruction parameters of at least one of the third monoenergetic image data or the fourth monoenergetic image data.

20. An image generating device, comprising:
a memory storing computer-executable instructions; and
at least one processor configured to execute the computer-executable instructions to cause the image generating device to
receive, in a first time interval, first spectrally differentiated CT projection measurement data that relates to a heart of a patient, wherein a contrast agent is located in at least one of chambers or vessels of the heart during the first time interval, and wherein the first time interval includes a diastole of the heart,
receive, in a second time interval, second spectrally differentiated CT projection measurement data that relates to the heart of the patient, wherein the contrast agent is located in a muscle tissue of the heart during the second time interval, and wherein the second time interval includes a systole of the heart,
calculate first monoenergetic image data for a first energy value based on the first spectrally differentiated CT projection measurement data,
calculate second monoenergetic image data for the first energy value based on the second spectrally differentiated CT projection measurement data,
calculate third monoenergetic image data for a second energy value based on the first spectrally differentiated CT projection measurement data,
calculate fourth monoenergetic image data for a third energy value based on the second spectrally differentiated CT projection measurement data, and
determine the second energy value and the third energy value such that an attenuation caused by the contrast agent in the third monoenergetic image data is aligned with an attenuation caused by the contrast agent in the fourth monoenergetic image data.
